(19) United States
(12) Patent Application Publication
(10) Pub. No.: US 2018/0110816 A1
(43) Pub. Date: Apr. 26, 2018

(54) Process For Producing A Fluid Soluble Cannabis Base Product

(71) Applicants: Peter Q Bum Suh, Hacienda Heights, CA (US); Song D. Suh, Hacienda Heights, CA (US)

(72) Inventors: Peter Q Bum Suh, Hacienda Heights, CA (US); Song D. Suh, Hacienda Heights, CA (US)

(21) Appl. No.: 15/790,970

(22) Filed: Oct. 23, 2017

Related U.S. Application Data
(60) Provisional application No. 62/411,664, filed on Oct. 23, 2016.

(51) Int. Cl. A61K 36/185; A61K 31/352; A61K 31/05

(52) U.S. Cl. CPC A61K 36/185; A61K 31/05; A61K 31/352

(57) ABSTRACT

A process for producing a fluid soluble cannabis base product that is used primarily to create an ingestible liquid with the benefits and effects of tetrahydrocannabinol (THC) and cannabidiol (CBD) in the cannabis. The process separates different cannabinoids in a cannabis plant and reconstitutes the cannabinoids into a fluid soluble solution having both the THC and CBD. By utilizing hydroxyl functional groups, cannabis plant material is converted into a fluid soluble solution. Then using reverse osmosis, remaining unmodified hydroxyl functional groups are removed through a semipermeable membrane acting as a filter. The remaining product is processed through four phases in which the product undergoes further thermos and kinetic modifications with hydrogen 2 oxygen 1 (H20). The end product is a fluid soluble or fluid compatible solution.

### TECHNICAL FIELD

The invention generally pertains to cannabis base products, and more particularly to a cannabis product that is fluid soluble and that can offer medicinal benefits with a selectable amount of the intoxicating effect.

### BACKGROUND ART

One of the most widely known and commonly used for multiple purposes plants is cannabis. Throughout the world cannabis has long been used as an intoxicant, for medical purposes, and to create tangible items such as hemp fibers which can be made into products such as clothing or rope.

In the preceding few decades, especially in developed countries, cannabis has become stigmatized as a result of its association with the drug marijuana. Only recently has marijuana, and as direct result cannabis, been accepted and used as an effective substance for medicinal purposes. In particular, marijuana has been especially effective when used by people who have various type of cancer and those with HIV/AIDS. Also, there has been a pronounced lessening in the public perception of marijuana as a harmful drug that can destroy the lives of those using it to get “high”.

Although marijuana/cannabis is effective as both an intoxicant and medicinal substance, there are problems inherent to its use. One problem is that many people do not want to smoke marijuana/cannabis and prefer to ingest it orally in the form of a food product (known as edibles). While edibles are capable of introducing marijuana/cannabis into a person, a typical edible will only allow a person to obtain the desired level of the tetrahydrocannabinol (THC) if accompanied by undesirable amounts of oil, fat and calories, all of which are unhealthy if taken in large amounts.

Also, many people who appreciate and require the medicinal benefits do not want to experience the “high” as well. The two major elements in marijuana/cannabis are THC and cannabidiol (CBD). The THC produces the intoxicating effect(s), and the CBD products much of the medicinal benefits(s), such as analgesic effects.

What is needed is a method of producing and offering a cannabis product in a form that does not provide un-wanted fat, oil or calories, and that can provide the desirable medicinal effects without the un-wanted intoxicating effect(s) of the THC. Optimally, the product would be fluid soluble or compatible, and could be offered with various amounts of THC, thereby allowing a user of the product the ability to choose how much of the intoxicating effect he/she wishes to experience with the medicinal benefits.

A search of the prior art did not disclose any literature or patents that read directly on the claims of the instant invention. However, the following U.S. patents are considered related:

| PATENT NO. | INVENTOR | ISSUED |
|---|---|---|
| 6,403,126 | Webster, et al | Jan. 24, 2000 |
| 8,337,908 | Letzel, et al | Sep. 26, 2008 |
| 2000/0049059 | Mueller | Oct. 16, 2003 |
| 2017/0157041 | Goldner | Jun. 8, 2017 |

The U.S. Pat. No. 6,403,126 patent discloses a method extracting cannabinoids, cannaflavins, and/or essential oils from hemp and/or of producing a whole hemp extract lacing THC. Industrial hemp is harvested and the chaff is threshed from the seeds. The chaff is then ground chaff is extracted with an organic solvent. The extract is then loaded onto a chromatographic column selected to fractionate specific cannabinoids, cannaflavins, and essential oils. In one embodiment, a whole hemp extract lacking THC is produced. In other embodiment, specific cannabinoids and related compounds are fractionated out, thereby producing purified cannabinoids, cannaflavins, and related compounds.

The U.S. Pat. No. 8,337,908 patent discloses a plant extract form a low-tetrahydrocannabinol (THC) variety of cannabis sativa substance for the treatment of disease. The invention further relates to the production of the extract and pharmaceutical compositions comprising the extract and the uses thereof.

The 2000/0049059 publication discloses a method for producing an extract from cannabis plant matter, containing tetrahydrocannabinol, cannabibiol and optionally the carboxylic acids thereof. According to the method, the dried plant matter is ground and subjected to a CO₂ extraction and the primary extract obtained is separated. The method permits or tetrahydrocannabinol to be selectively obtained both from industrial hemp and from drug-producing hemp, optionally after dissolving the primary extract in ethanol, separating undesirable waxes and removing the solvent under reduced pressure.

The 2017/0157041 publication discloses an orally dissolvable cannabis table that includes one or more active cannabis-based chemical constituents and one or more inactive constituents. The active cannabis-based chemical constituents includes at least one of cannabinoids or terpenoids.

For background purposes and indicative of the art to which the invention relates reference may be made to the following remaining patents found in the patent search.

| PATENT NO. | INVENTOR | ISSUED |
|---|---|---|
| 5,847,128 | Martin, et al | Dec. 8, 1998 |
| 9,066,910 | Rosenblatt, et al | Jun. 30, 2015 |
| 9,480,647 | Benson, et al | Nov. 1, 2016 |
| 9,629,886 | Franklin, et al | Apr. 25, 2017 |
| 9,694,040 | Sciadone | Jul. 4, 2017 |

### DISCLOSURE OF THE INVENTION

A process for producing a fluid soluble cannabis base product. The process separates different cannabinoids in a cannabis plat and reconstructs the cannabinoids into a fluid soluble solution having both tetrahydrocannabinol (THC) and cannabidiol (CBD). By utilizing hydroxyl functional groups, cannabis plant material is converted into a fluid soluble solution. Reverse osmosis is then used to remove remaining unchanged hydroxyl functional groups through a semi-permeable membrane, which acts as a molecular filter. The remaining product is processed through phases in which the product undergoes further thermos and kinetic modification with hydrogen 2 oxygen 1 (H20). The end product is a fluid soluble or fluid compatible solution.

The steps for producing the fluid soluble cannabis base product are:

1) Acquire and prepare a quantity of cannabis plant material.

2) Expose the cannabis plant material to nitrogen, without any physical contact of the nitrogen to the plant, to reduce the plant material's core temperature to between −20° −80°, with −80° optimal.

3) Place cooled cannabis plant material into waterproof/heat proof netting.

4) Expose cannabis plant material to a temperature ranging from 235° F. to 250° F. with 250° F. optimal, and pressure ranging from 2000 tons to 20 tons with 20 tons optimal thereby producing what is known as essence of plant material (EPM).

5) Apply EPM to hydroxyl functional groups, which are a base of carbon and hydrogen molecules set to interact with EPM.

6) Apply EPM to four phases of amplitude processing, with each phase increasing in temperature.

7) Apply reverse-osmosis to filter molecular impurities, thereby resulting in the fluid soluble cannabis product.

8) Insert fluid soluble cannabis product into a container for storage or to facilitate use.

The fluid soluble cannabis base product is primarily utilized to produce orally ingestible liquid with the benefits and effects of THC and CBD. The cannabis base product can also be used to produce other items such as dermally-applied cremes that are effective for reducing pain such as for arthritis, or a variety of liquid based food items.

In view of the above disclosure, the primary object of the invention is to provide a Process for producing a fluid soluble base product that can be used to create a liquid that can be ingested by a person.

In addition to the primary object, it is also an object of the invention to provide a process for producing a fluid soluble base product that:
is easy to use,
can be utilized as a base product for a variety of items,
is easy to manufacture.
provides significant health benefits,
removes the need for smoking as a method of ingesting cannabis,
can be made as THC only, CBD only, or a combination of THC and CBD,
delivers a potent dose of THC with the accompanying effects.
is legal in multiple states, and
is cost effective from both a manufacturers' and consumer's point of view.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a flow down block diagram of the steps of the process.

FIG. 2 is a diagram showing phase I of the four phases processing that produces thermo and kinetic changes with H20.

FIG. 3 is a diagram showing phase II of the four phases processing that produces thermo and kinetic changes with H20.

FIG. 4 is a diagram showing phase III of the four phases processing that produces thermo and kinetic changes with H20.

FIG. 5 is a diagram showing phase IV of the four phases processing that produces thermo and kinetic changes with H20.

### BEST MODE FOR CARRYING OUT THE INVENTION

The best mode for carrying out the invention is presented in terms that disclose a preferred embodiment of a process for producing a fluid soluble cannabis product (the process 10).

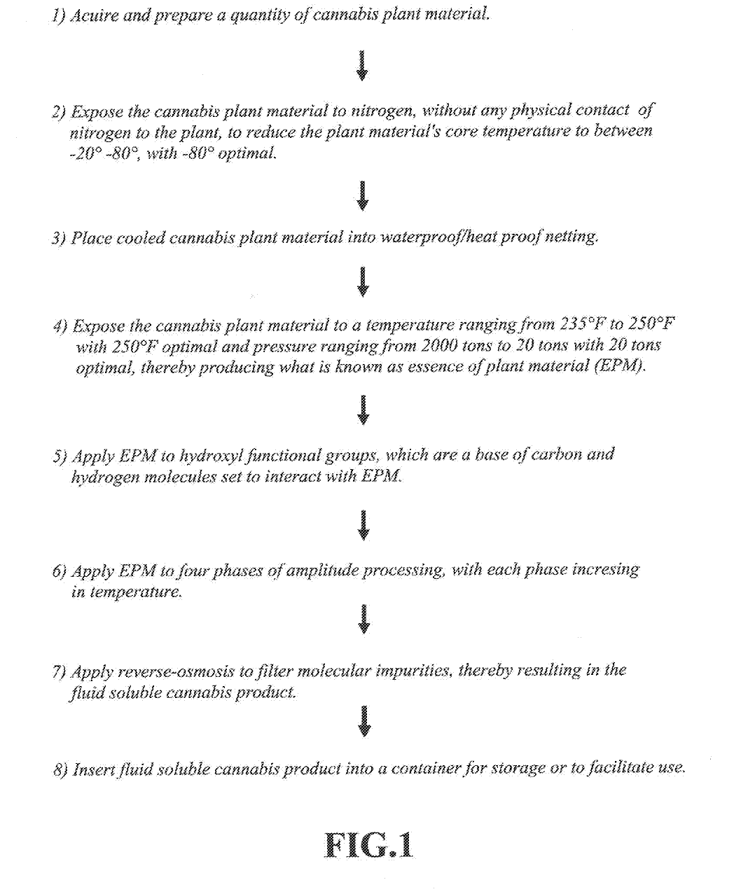

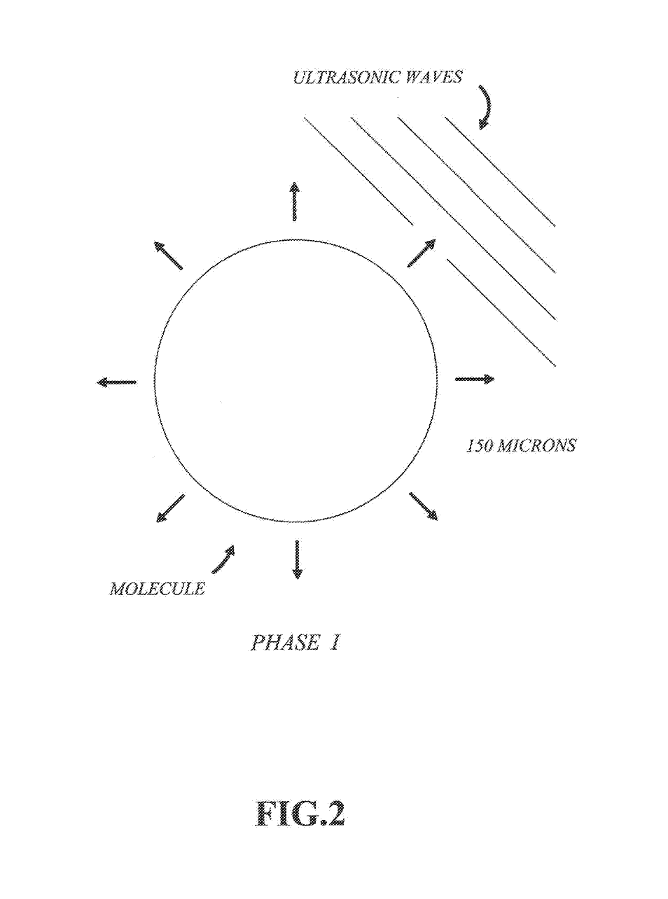

The process 10, as shown in FIGS. 1 and 2, is designed to separate different cannabinoids in a cannabis plant and reconstructing the cannabinoids into a formulation that is medically viable. The formulation that is produced modifies a conventional cannabis plant into a fluid soluble solution having both Tetrahydrocannabinol (THC) and Cannabidiol (CBD), as well as vitamins and natural sugars.

Besides the traditional means of cannabis administration (inhalation), the current cannabis market is heavily saturated with oil based products. This is because oil forms are the easiest way to derive THC from a cannabis plant. The advantages of creating a fluid soluble THC and CBD product that does not produce oil and fat are significant. Producing a fluid soluble form of cannabis plant that does not rely on fat increases the rate of absorption into a persons's body, resulting in a much shorter activation time.

CBD is a non-psychoactive, highly therapeutic compound that is present in cannabis. THC binds to CB1 while CBD has a greater affinity to bind to CB1 receptors (receptors in the central nervous system) and CB2 receptors (receptors in the immune system). CBD provides major health benefits including:
Antiemetic, anticonvulsant, antipsychotic, anti-inflammatory, antioxidant, antitumoral, and anti olytic. Most importantly CBD act as a neuroprotective agent, supporting the findings of neuroplasticity.

The process 10, as shown in FIG. 1, not only extracts 99% of all cannabinoids but also over a dozen specific hydrocarbons known as terpenes which not only give cannabis its distinct aroma and flavor but alter the “high” itself. The cannabinoids and terpenes interact cooperatively to create what referred to as an “entourage effect” that magnifies the therapeutic benefits of the cannabis plant's individual components so that the medicinal impact of the whole plant is greater than the sum of its parts.

CBD also has an effect on the production and release of neurotransmitters. Cannabis plant derived CBD stimulates the production and release of neurotransmitters, thus regulating homeostasis. CBD at high dosages of 20 mg or more have been shown to effectively stimulate both CB1 and CB2 receptors in the human body, which are involved with the immune system; effecting functions such as immune suppression or apoptosis (programmed cell death of cancer cells). CBD modulates the pain sensation as well as various diseases, from liver to neurodegenerative diseases. A higher concentration of CBD has been shown to activates 5-HT1A serotonin receptors, producing antidepressant effects. CBD also blocks CPR 55 signaling, decreasing bone resorption and cancer cell proliferation. The activation of adenosine receptors by CBD gives it the anti-anxiety and anti-inflammatory effects of cannabidiol. The adenosine receptors release dopamine and glutamate and two adenosine receptors release dopamine and glutamate which are two essential neurotransmitters that decrease during the course of a person's life span.

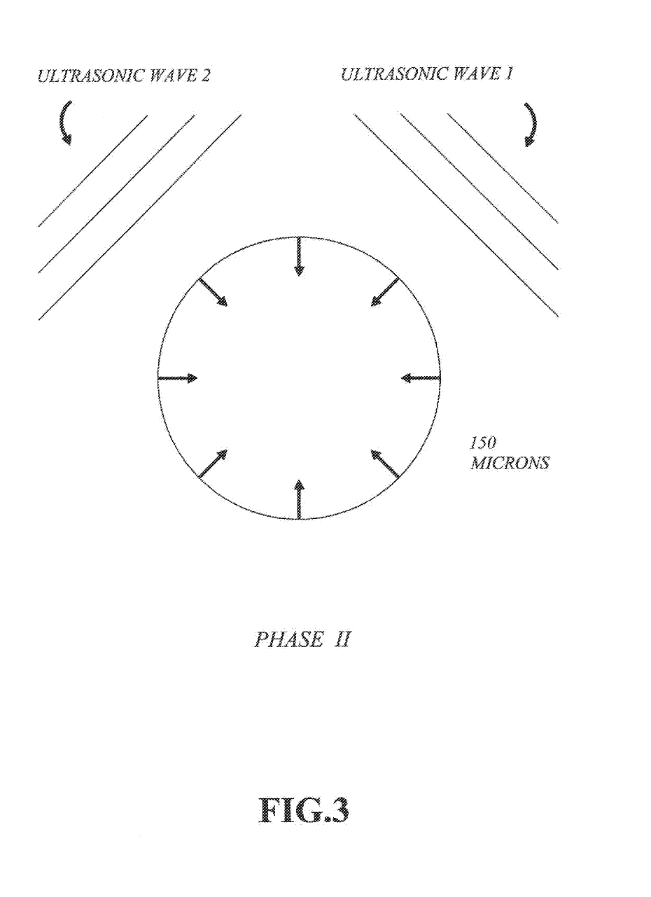

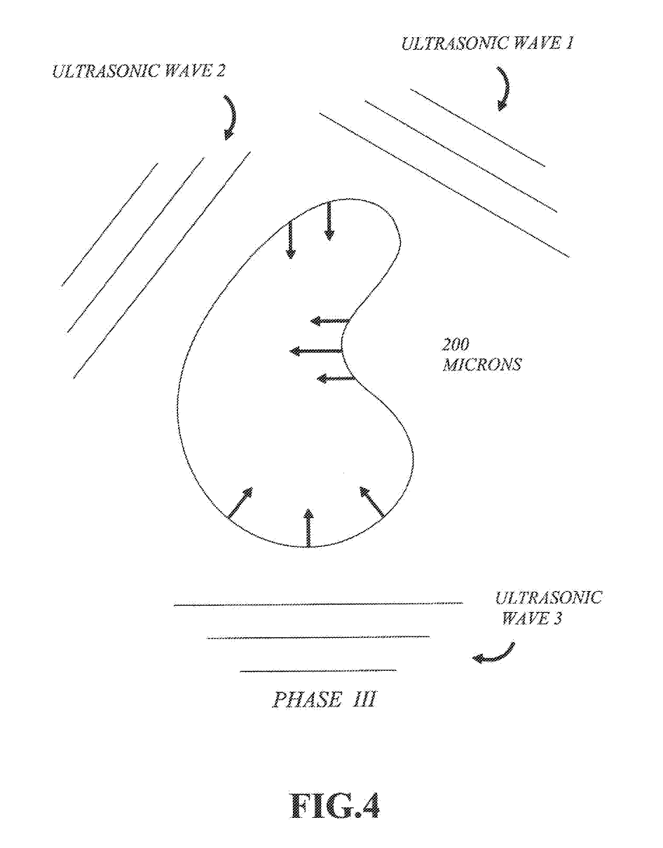

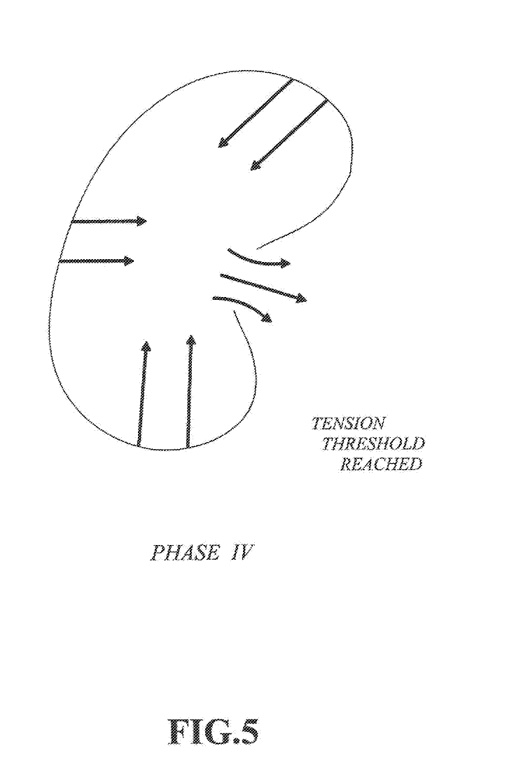

Under specific thermos and kinetic conditions, with the utilization of hydroxyl functional groups, cannabis plant material can be converted into a fluid soluble or fluid compatible solution. Then using a form of reverse osmosis the remaining unchanged hydroxyl functional groups are removed through a semipermeable membrane acting as a molecular filter. The remaining product is processed through four phases in which the product undergoes further thermo and kinetic changes with hydrogen 2 oxygen 1 (H20). In Phase 1, as shown in FIG. 2, a molecule is exposed to one group of ultrasonic waves. In Phase 2, as shown in FIG. 3, the molecule is exposed to two groups of ultrasonic waves. In Phase 3, as shown in FIG. 4, the molecule is exposed to three groups of ultrasonic waves. And in Phase 4, as shown in FIG. 5, the molecule's tension threshold is reached.ioujlkjlo’ The end product is a fluid soluble or fluid compatible solution.

The cannabis plant material must first be acquired and then prepared to endure pressure and heat for an extended period of time. Using liquid nitrogen as a cooling agent the plant material is subjected to temperatures that freeze the cell walls of the plant. The cannabis plant trichomes, where most of the THC is located, become brittle and more susceptible to heat and pressure. The brittleness of the cannabis material, as well as material on the cannabis plant, is key in introducing the cannabis plant to the required high pressure.

The cannabis plant is separated into multiple parts and the parts are placed inside waterproof/heat proof netting. This is to ensure that any material that comes out of the cannabis plant can be separated from the cannabis plant cell material. The extreme pressure and heat that the plant will be exposed to secretes material that is separated from the cannabis plant material.

After the cannabis plant material is secured within the netting, pressure ranging from 20 tons to 2000 tons, with 20 tons preferred, at a temperature ranging from 200° F. to 275° F., with 250° F. preferred is applied. The application of pressure and change of heat alters the kinetic stability of the cannabis plant material to its lowest form of energy, thereby preventing the material from changing states for extended periods of time and creating what is called the essence of the cannabis plant material which is referred to as EPM and is added to a specific hydroxyl functional group. From here the EPM undergoes a change that will allow the EPM to break into smaller molecules with the hydroxyl functional groups.

The EPM and hydroxyl substance is then exposed to extreme cold temperatures ranging from −100° C. to −42° C., as well as high amplitude processing, set into four phases, as shown in FIG. 2. The high amplitude processing utilizes micron waves that are emitted and function to break-up the molecular tension in the structure of the EPM and hydroxyl substances. This is to ensure the newly created substance has thermodynamic stability while slowly exchanging hydroxyl groups with Hydrogen2 Oxygen. The exposure of the EPM and hydroxyl group to the above process displaces the EPM molecules within the hydroxyl groups. Then reverse osmosis is used to filter molecular impurities as well as unnecessary hydroxyl groups, producing a fluid soluble or fluid compatible liquid substance. It should be noted that while the process 10 is primarily utilized to produce a base product for a fluid soluble liquid that can be orally ingested, other items can also be derived from the base product including dermally-applied crème for pain such as arthritis, or a liquid-based food item.

The steps, as shown in FIG. 1, of the process for producing a fluid soluble cannabis product are:

1) Acquire and prepare a quantity of cannabis plant material.

2) Expose the cannabis plant material to nitrogen, without any physical contact of the nitrogen to the plant, to reduce the plant material's core temperature to between −20° −80°, with −80° optimal.

3) Place cooled cannabis plant material into waterproof/heat proof netting.

4) Expose cannabis plant material to a temperature ranging from 235° F. to 250° F. with 250° F. optimal, and pressure ranging from 2000 tons to 20 tons with 20 tons optimal, thereby producing what is known as essence of plant material (EPM).

5) Apply EPM to hydroxyl functional groups, which are a base of carbon and hydrogen molecules set to interact with EPM.

6) Apply EPM to four phases of amplitude processing, with each phase increasing in temperature.

7) Apply reverse-osmosis to filter molecular impurities, thereby resulting in the fluid soluble cannabis product.

8) Insert fluid soluble cannabis product into a container for storage or to facilitate use.

While the invention has been described in detail and pictorially shown in the accompanying drawings it is not to be limited to such details, since many changes and modifications may be made to the invention without departing for the spirit and the scope thereof. Hence, it is described to cover any and all modifications and forms which may come within the language and scope of the claims.

1. A process for producing a fluid soluble cannabis base product, wherein said process separates different cannabinoids in a cannabis plant and reconstructs the cannabinoids into a fluid soluble solution having both tetrahydrocannabinol (THC) and cannabidiol (CBD), wherein utilizing hydroxyl functional groups, cannabis plat material is converted into a fluid soluble solution, wherein then using reverse osmosis, remaining unchanged hydroxyl functional groups are removed through a semipermeable membrane acting as a molecular filter, wherein remaining product is processed through phases in which the product undergoes further thermo and kinetic modifications with hydrogen 2 oxygen1 (H20), wherein an end product is a fluid soluble or fluid compatible solution.

2. The process for producing a fluid soluble cannabis base product as specified in claim 1 wherein said process is utilized to produce a fluid soluble base product from organic material.

3. The process for producing a fluid soluble cannabis base product as specified in claim 1 wherein said cannabis base product is produced without utilizing fat-derived oil-based products, wherein said cannabis base product not utilizing oil-based products increases the rate of absorption into a person's body, resulting in a shorter activation time compared to oil-based products.

4. The process for producing a fluid soluble cannabis base product as specified in claim 1 wherein said CBD is a non-psychoactive therapeutic compound which provides health benefits that are selected from the group consisting of antiemetic, anticonvulsant, antipsychotic, anti-inflammatory, antioxidant, antitumoral, anti-olytic and as a neuroprotective agent supporting the finds of neuroplasticity.

5. The process for producing a fluid soluble cannabis base product as specified in claim 1 wherein said CBD stimulates the production and release of neurotransmitters, thus regulating homeostasis.

6. The process for producing a fluid soluble cannabis base product as specified in claim 1 wherein said CBD stimulates CB1 and CB2 receptors in a human body, thereby effecting the human immune system and functions including immune suppression and cancer cell proliferation.

7. A process for producing a fluid soluble cannabis base product as specified in claim 1 wherein said process separates different cannabinoids in a cannabis plant and reconstructs the cannabinoids into a fluid soluble solution having both tetrahydrocannabinol (THC) and cannabidiol (CBD), wherein utilizing hydroxyl functional groups, cannabis plat material is converted into a fluid soluble solution, wherein then using reverse osmosis, remaining unchanged hydroxyl functional groups are removed through a semipermeable membrane acting as a molecular filter, wherein remaining product is processed through phases in which the product undergoes further thereto and kinetic changes with hydrogen 2 oxygen1 (H20), wherein an end product is a fluid soluble or fluid compatible solution wherein said process comprises the following steps:
a) acquire a quantity of cannabis plant material,
b) prepare said cannabis plant material to endure pressure and heat for extended periods of time,
c) use liquid nitrogen as a cooling agent that causes the plant material to be subjected to temperatures that freeze cell walls of the plant, thereby resulting in the cannabis plant's trichomes, where the majority of THC is located, to become brittle and more susceptible to heat and pressure,
d) separate the plant material into multiples parts,
e) place the multiple parts into waterproof and heat resistant netting, thereby ensuring that material released from the cannabis plant is separated from the cannabis plant cell material,
f) apply pressure ranging from 20 tons to 2000 tons, at a temperature ranging from 200° F. to 275° F., wherein the application of pressure and heat alters the kinetic stability of the plant material to a lowest form of energy, thereby preventing the plant material from changing states for extended periods of time, wherein after the pressure and heat is applied a product called essence of plant material (EPM) is created,
g) add the EPM to a substance known as a hydroxyl functional group, from wherein the EPM undergoes a change that allows the EPM to break into smaller molecules with the hydroxyl functional group,
h) expose the EPM and hydroxyl substance to cold temperatures ranging from −100° C. to −42° C., as well as high amplitude processing set into four phases, wherein the high amplitude processing utilizes micron waves that are emitted and function to break-up the molecular tension in the structure of the EPM and hydroxyl substance, wherein this is to ensure the newly created substance has thermodynamic stability while slowly exchanging hydroxyl groups with H20, wherein the exposure of the EPM and hydroxyl group to the above process displaces the EPM molecules within the hydroxyl groups, and
i) apply reverse osmosis to further remove molecular impurities as well as unnecessary hydroxyl groups, thereby producing a fluid soluble or fluid compatible liquid substance.

8. The process for producing a fluid soluble cannabis base product as specified in claim 7 wherein said process is utilized to produce a fluid soluble base product from organic material.

9. The process for producing a fluid soluble cannabis base product as specified in claim 7 wherein said cannabis base product is produced without utilizing fat-derived oil-based products, wherein said cannabis base product not utilizing oil-based products increases the rate of absorption into a person's body, resulting in a shorter activation time compared to oil-based products.

10. The process for producing a fluid soluble cannabis base product as specified in claim 7 wherein said CBD is a non-psychoactive therapeutic compound which provides health benefits that are selected from the group consisting of antiemetic, anticonvulsant, antipsychotic, anti-inflammatory, antioxidant, antitumoral, anti-olytic and as a neuroprotective agent supporting the finds of neuroplasticity.

11. The process for producing a fluid soluble cannabis base product as specified in claim 7 wherein said CBD stimulates the production and release of neurotransmitters, thus regulating homeostasis.

12. The process for producing a fluid soluble cannabis base product as specified in claim 7 wherein said CBD stimulates CB1 and CB2 receptors in a human body, thereby effecting the human immune system and functions including immune suppression and cancer cell proliferation.

13. The process for producing a fluid soluble cannabis base product as specified in claim 7 wherein cannabinoids and specific hydrocarbons known as terpenes interact cooperatively to create what is referred to as an “entourage effect” that magnifies the therapeutic benefits of the cannabis plant's individual components so that the medicinal impact of the whole plant is greater than the sum of its parts.

14. The process for producing a fluid soluble cannabis base product as specified in claim 7 wherein said CBD activates 5-HT1A serotonin receptors to produce antidepressant effects, and activates adenosine receptors to product anti-anxiety and anti-inflammatory effects.

15. A process for producing a fluid soluble cannabis base product wherein said process separates different cannabinoids in a cannabis plant and reconstructs the cannabinoids into a fluid soluble solution having both tetrahydrocannabinol (THC) and cannabidiol (CBD), wherein utilizing hydroxyl functional groups, cannabis plat material is converted into a fluid soluble solution, wherein then using reverse osmosis, remaining unchanged hydroxyl functional groups are removed through a semipermeable membrane acting as a molecular filter, wherein remaining product is processed through phases in which the product undergoes further thereto and kinetic changes with hydrogen 2 oxygen1 (H20), wherein an end product is a fluid soluble or fluid compatible solution, wherein the process is comprised of the following steps;
1) Acquire and prepare a quantity of cannabis plant material,
2) Expose the cannabis plant material to nitrogen, without any physical contact of the nitrogen to the plant, to reduce the plant material's core temperature to between −20° −80°, with −80° optimal,
3) Place cooled cannabis plant material into waterproof/heat proof netting,
4) Expose cannabis plant material to a temperature ranging from 200° F. to 275° F. with 250° F. optimal, and pressure ranging from 20 tons to 2000 tons with 20 tons optimal, thereby producing what is known as essence of plant material (EPM),
5) Apply EPM to hydroxyl functional groups, which are a substance comprising a base of carbon and hydrogen molecules set to interact with the EPM,
6) Apply EPM to four phases of amplitude processing, with each phase increasing in temperature,
7) Apply reverse-osmosis to filter molecular impurities, thereby resulting in the fluid soluble cannabis product, and
8) Insert fluid soluble cannabis product into a container for storage or to facilitate use.

16. The process for producing a fluid soluble cannabis base product as specified in claim 15 wherein said cannabis base product is produced without utilizing fat-derived oil-based products, wherein said cannabis base product not utilizing oil-based products increases the rate of absorption into a person's body, resulting in a shorter activation time compared to oil-based products.

17. The process for producing a fluid soluble cannabis base product as specified in claim 15 wherein said SBD is a non-psychoactive therapeutic compound which provides health benefits that are selected from the group consisting of antiemetic, anticonvulsant, antipsychotic, anti-inflammatory, antioxidant, antitumoral, anti-olytic and as a neuroprotective agent supporting the finds of neuroplasticity.

18. The process for producing a fluid soluble cannabis base product as specified in claim 15 wherein said CBD stimulates the production and release of neurotransmitters, thus regulating homeostasis.

19. The process for producing a fluid soluble cannabis base product as specified in claim 15 wherein said CBD stimulates CB1 and CB2 receptors in a human body, thereby effecting the human immune system and functions including immune suppression and cancer cell proliferation.

20. The process for producing a fluid soluble cannabis base product as specified in claim 15 wherein cannabinoids and specific hydrocarbons known as terpenes interact cooperatively to create what referred to as an “entourage effect” that magnifies the therapeutic benefits of the cannabis plant's individual components so that the medicinal impact of the whole plant is greater than the sum of its parts.

21. The process for producing a fluid soluble cannabis base product as specified in claim 15 wherein said CBD activates 5-HT1A serotonin receptors to produce antidepressant effects, and activates adenosine receptors to product anti-anxiety and anti-inflammatory effects.
